(19) United States
(12) Patent Application Publication
(10) Pub. No.: US 2025/0252571 A1
(43) Pub. Date: Aug. 7, 2025

(54) SYSTEM AND METHOD FOR AUTOMATIC VOLUME OF INTEREST PRESCRIPTION FOR MULTI-VOXEL BRAIN PROTON SPECTROSCOPY ACQUISITION AND POST PROCESSING

(71) Applicant: GE Precision Healthcare LLC, Waukesha, WI (US)

(72) Inventors: Sajith Rajamani, Bangalore (IN); Florintina Chaarlas, Bangalore (IN); Naga Gayathri Matcha, Vijayawada (IN); Rajagopalan Sundaresan, Bangalore (IN)

(21) Appl. No.: 18/431,429

(22) Filed: Feb. 2, 2024

(51) Int. Cl. G06T 7/00; A61B 5/00; A61B 5/055; G06T 7/11; G06T 7/62; G06T 11/20; G06V 10/25

(52) U.S. Cl. CPC G06T 7/0014; A61B 5/0042; A61B 5/055; G06T 7/11; G06T 7/62; G06T 11/206; G06V 10/25; G06T 2207/10088; G06T 2207/20081; G06T 2207/20092; G06T 2207/30016; G06T 2207/30096

(57) ABSTRACT

A method for performing multi-voxel spectroscopy includes obtaining structural magnetic resonance imaging data of a brain of a subject acquired with a magnetic resonance imaging scanner. The method also includes performing skull stripping on the structural magnetic resonance imaging data to generate a skull stripped brain image. The method further includes utilizing a trained deep learning-based segmentation model to generate a lesion core mask from the brain image. The method also includes locating a slice with largest volume of lesion present in the lesion core mask. The method includes calculating a voxel volume that avoids aliasing from the slice based on a field of view. The method includes automatically selecting a volume of interest in the brain having both the lesion and normal brain tissue for a multi-voxel spectroscopy scan by the magnetic resonance imaging scanner based on the brain image, the lesion core mask, and the voxel volume.

### BACKGROUND

The subject matter disclosed herein relates to medical imaging and, more particularly, to a system and a method for automatic volume of interest prescription for multi-voxel brain proton spectroscopy acquisition and post processing.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging (MRI), when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Proton (¹H) spectroscopy is an import sequence in MRI brain tumor imaging to study the metabolites and to differentiate between neoplastic and non-neoplastic lesions. Currently, there are two types of spectroscopic acquisition available: single voxel and multi-voxel techniques. When utilizing the single voxel technique, the user can acquire data from one region of interest in the brain. When utilizing the multi-voxel technique, the user can acquire a large volume of interest. But acquiring this large volume of interest involves additional post processing to generate individual spectroscopic graphs. The major advantage of the multi-voxel technique is that metabolic information from the normal brain area can also be calculated from the acquired data and the user can compare the metabolites information of the lesion with the normal brain tissue. A major challenge in the acquisition of good spectroscopic data is planning the volume of interest. The operator needs to interpret all the structural sequences used to identify the lesion and then to choose the appropriate slice for the acquisition of the multi-voxel spectroscopy volume of interest that should include the abnormality as well as adjacent or contralateral normal brain tissues. Care should be taken to ensure that the volume of interest does not include bone or air-tissue interface since including this would cause degradation of the spectroscopy data because of B₀ inhomogeneity in the interfaces. Due to this, a high level of expertise is required in planning the multi-voxel spectroscopic sequence and in post processing of the multi-voxel spectroscopy. This expertise extends to interpretation of whole structural data to find the abnormality and selection of appropriate slice from the structural sequences (e.g., T1, T2, T2 Flair, T1 post contrast, etc.). Inadequate magnetic resonance spectroscopy planning will degrade the metabolites graph which is required to differentiate various brain lesions and ultimately result in re-scanning of patient.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing multi-voxel spectroscopy is provided. The computer-implemented method includes obtaining, at a processor, structural magnetic resonance imaging data of a brain of a subject acquired with a magnetic resonance imaging scanner. The computer-implemented method includes performing, via the processor, skull stripping on the structural magnetic resonance imaging data to generate a skull stripped brain image. The computer-implemented method further includes utilizing, via the processor, a trained deep learning-based segmentation model to generate a lesion core mask from the skull stripped brain image. The computer-implemented method even further includes locating, via the processor, a slice with largest volume of lesion present in the lesion core mask. The computer-implemented method still further includes calculating, via the processor, a voxel volume that avoids aliasing from the slice based on a field of view. The computer-implemented method yet further includes automatically selecting, via the processor, a volume of interest in the brain having both the lesion and normal brain tissue for a multi-voxel spectroscopy scan by the magnetic resonance imaging scanner based on the skull stripped brain image, the lesion core mask, and the voxel volume.

In another embodiment, a computer-implemented method for post-processing for multi-voxel spectroscopy is provided. The computer-implemented method includes obtaining, at a processor, susceptibility-weighted image data of a brain of a subject acquired during a multi-voxel spectroscopy scan with a magnetic resonance imaging scanner. The computer-implemented method also includes performing, via the processor, skull stripping on the susceptibility-weighted image data to generate a skull stripped brain image. The computer-implemented method further includes utilizing, via the processor, a trained deep learning-based segmentation model to generate a bleeds and calcification mask, a lesion core mask, and a brain mask with no abnormality from the skull stripped brain image. The computer-implemented method even further includes automatically locating, via the processor, within a prescribed volume of interest the largest voxel with pathology devoid of bleeds and calcification based on both the lesion core mask and the bleeds and calcification mask, wherein the prescribed volume of interest has both a lesion and normal brain tissue. The computer-implemented method still further includes automatically locating, via the processor, within the prescribed volume of interest a corresponding voxel of a healthy region of the brain based on both the brain mask with no abnormality and the bleeds and calcification mask. The computer-implemented method yet further includes providing, via the processor, a request to user to finalize selection of voxels including the largest voxel with pathology devoid of bleeds and calcification and the corresponding voxel of the healthy region. The computer-implemented method further includes generating, via the processor, upon finalization of selection of voxels, respective metabolites graphs for both the largest voxel with pathology devoid of bleeds and calcification and the corresponding voxel of the healthy region.

In another embodiment, a system for performing multi-voxel spectroscopy is provided. The system includes a memory encoding processor-executable routines. The system also includes a processor configured to access the memory and to execute the processor-executable routines, wherein the routines, when executed by the processor, cause the processor to perform actions. The actions include obtaining structural magnetic resonance imaging data of a brain of a subject acquired with a magnetic resonance imaging scanner. The actions also include performing skull stripping on the structural magnetic resonance imaging data to generate a skull stripped brain image. The actions further include utilizing a trained deep learning-based segmentation model to generate a lesion core mask from the skull stripped brain image. The actions even further include locating a slice with largest volume of lesion present in the lesion core mask. The actions still further includes calculating a voxel volume that avoids aliasing from the slice based on a field of view. The actions yet further include automatically selecting a volume of interest in the brain having both the lesion and normal brain tissue for a multi-voxel spectroscopy scan by the magnetic resonance imaging scanner based on the skull stripped brain image, the lesion core mask, and the voxel volume.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates a schematic diagram of a magnetic resonance imaging (MRI) system suitable for use with the disclosed techniques;

FIG. 2 illustrates a flow diagram of a method for performing multi-voxel spectroscopy, in accordance with aspects of the present disclosure;

FIG. 3 is a schematic diagram illustrating the process for prescribing a volume of interest for multi-voxel spectroscopy, in accordance with aspects of the present disclosure;

FIG. 4 illustrates a flow diagram of a method for selecting a volume of interest, in accordance with aspects of the present disclosure;

FIG. 5 illustrates utilization of a volume of interest selection algorithm (e.g., utilizing a horizontal volume), in accordance with aspects of the present disclosure;

FIG. 6 illustrates utilization of a volume of interest selection algorithm (e.g., utilizing a vertical volume), in accordance with aspects of the present disclosure;

FIG. 7 illustrates a flow diagram of a method for post-processing for multi-voxel spectroscopy, in accordance with aspects of the present disclosure;

FIG. 8 is a schematic diagram illustrating the process for post-processing for multi-voxel spectroscopy, in accordance with aspects of the present disclosure;

FIG. 9 illustrates display of a metabolites graph and corresponding location for voxel placed in contrast enhanced portion of pathology, in accordance with aspects of the present disclosure; and

FIG. 10 illustrates display of a metabolites graph and corresponding location for a contralateral voxel in a healthy brain region, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Deep learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and_{/}or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for performing multi-voxel spectroscopy. In particular, the present disclosure provides systems and methods for automatically identifying and selecting a slice has a lesion from structural magnetic resonance imaging data (e.g., T1, T2, T2 Flair, T1 post contrast, etc.) and prescribing a volume of interest that includes both the abnormality and adjacent or contralateral brain tissue (excluding the bone and air-tissue interfaces). The exclusion of the bone and air-tissue interfaces reduces magnetic field inhomogeneity (which could degrade the metabolites graph quality).

For example, the disclosed systems and methods include obtaining, at a processor, structural magnetic resonance imaging data (e.g., T1-weighted image, T2-weighted image, fluid-attenuated inversion recovery (FLAIR) image, T1 weighted image post contrast) of a brain of a subject acquired with a magnetic resonance imaging scanner. The disclosed systems and methods also include performing, via the processor, skull stripping on the structural magnetic resonance imaging data to generate a skull stripped brain image. The disclosed systems and methods further include utilizing, via the processor, a trained deep learning-based segmentation model to generate a lesion core mask from the skull stripped brain image. The disclosed systems and methods even further include locating, via the processor, a slice with largest volume of lesion present in the lesion core mask. The disclosed systems and methods still further include calculating, via the processor, a voxel volume that avoids aliasing from the slice based on a field of view. The disclosed systems and methods also include automatically selecting, via the processor, a volume of interest in the brain having both the lesion and normal brain tissue for a multi-voxel spectroscopy scan by the magnetic resonance imaging scanner based on the skull stripped brain image, the lesion core mask, and the voxel volume.

In certain embodiments, automatically selecting the volume of interest includes identifying, via the processor, in which hemisphere of the brain that a core of the lesion is located utilizing the brain mask. In certain embodiments, automatically selecting the volume of interest further includes determining, via the processor, in the brain tissue whether to utilize a horizontal volume of interest or vertical volume of interest.

In certain embodiments, brain tissue mask is used to determine whether to utilize the horizontal volume of interest or the vertical volume of interest. This process includes studying, via the processor, a plot of a horizontal line as thick as the voxel volume that both originates from the brain tissue mask from the hemisphere of the brain where the core of the lesion is located and passes through a center of the core until an end of the brain tissue mask to determine if intensity decreases along the horizontal line. In certain embodiments, determining in the brain tissue mask to utilize the horizontal volume of interest or the vertical volume of interest further includes determining, via the processor, that the horizontal volume of interest can be utilized when the intensity does not decrease along the horizontal line. In certain embodiments, determining in the brain tissue mask to utilize the horizontal volume of interest or the vertical volume of interest further includes rotating, via the processor, the horizontal line 90 degrees to become a vertical line when the intensity does decrease along the horizontal line. In certain embodiments, determining in the brain tissue mask to utilize the horizontal volume of interest or the vertical volume of interest further includes determining, via the processor, if the intensity decreases along the vertical line. In certain embodiments, determining in the brain tissue mask to utilize the horizontal volume of interest or the vertical volume of interest further includes determining, via the processor, that the vertical volume of interest can be utilized when the intensity does not decrease along the vertical line.

In certain embodiments, the disclosed system and methods include providing, via the processor, a notification recommending a single voxel spectroscopy scan of the brain instead of a multi-voxel spectroscopy scan when the intensity decreases along both the horizontal line and the vertical line. In certain embodiments, automatically selecting the volume of interest further includes determining, via the processor, a number of voxels to be utilized in the volume of interest. In certain embodiments, the disclosed systems and methods include causing, via the processor, acquisition of susceptibility-weighted image data of the brain of the subject for the volume of interest during a multi-voxel spectroscopy scan with the magnetic resonance imaging scanner.

The disclosed systems and methods include obtaining, at a processor, susceptibility-weighted image data of a brain of a subject acquired during a multi-voxel spectroscopy scan with a magnetic resonance imaging scanner. The disclosed systems and methods also include performing, via the processor, skull stripping on the susceptibility-weighted image data to generate a skull stripped brain image. The disclosed systems and methods further include utilizing, via the processor, a trained deep learning-based segmentation model to generate a bleeds and calcification mask, a lesion core mask, and a brain mask with no abnormality from the skull stripped brain image. The disclosed systems and methods even further include automatically locating, via the processor, within a prescribed volume of interest the largest voxel with pathology devoid of bleeds and calcification based on both the lesion core mask and the bleeds and calcification mask, wherein the prescribed volume of interest has both a lesion and normal brain tissue. The disclosed systems and methods still further include automatically locating, via the processor, within the prescribed volume of interest a corresponding voxel of a healthy region of the brain based on both the brain mask with no abnormality and the bleeds and calcification mask. The disclosed systems and methods yet further include providing, via the processor, a request to user to finalize selection of voxels including the largest voxel with pathology devoid of bleeds and calcification and the corresponding voxel of the healthy region. In certain embodiments, the request includes an option presented to the user to edit or to add more voxels. The disclosed systems and methods further include generating, via the processor, upon finalization of selection of voxels, respective metabolites graphs for both the largest voxel with pathology devoid of bleeds and calcification and the corresponding voxel of the healthy region. In certain embodiments, the disclosed systems and methods include causing display of both the respective metabolites graphs and respective locations of both the largest voxel with pathology devoid of bleeds and calcification and the corresponding voxel of the healthy region on a reference image of the brain of the subject utilized for prescription of the volume of interest for the multi-voxel spectroscopy scan.

The disclosed embodiments help the user detect pathology and select a slice for spectroscopy planning. The disclosed embodiments also reduce planning time and avoid unnecessary re-scanning of spectroscopy due to lack of expertise for first time magnetic resonance imaging users. The disclosed embodiments help avoid intra-voxel magnetic field inhomogeneity artifacts by excluding the bone and air-tissue interface while prescribing the spectroscopy volume of interest. The disclosed embodiments enable the most appropriate voxels to be chosen automatically for studying, thus, helping with the magnetic resonance imaging reporting.

Although the techniques described below are utilized for acquiring multi-voxel spectroscopy of brain tumors, the techniques can be utilized for multi-voxel spectroscopy of other pathologies. For example, the disclosed techniques may be utilized for bilateral hippocampal spectroscopy acquisition in cases of epilepsy. The disclosed techniques may also be utilized for metabolic disorders, demyelinating disorders, neurodegenerative disease, and other applications. For example, the user may choose an area of interest form a drop-down menu for utilizing the techniques (e.g., hippocampus for epilepsy, parietal cortex or basal ganglia in case of suspected metabolic disorders of the brain, etc.).

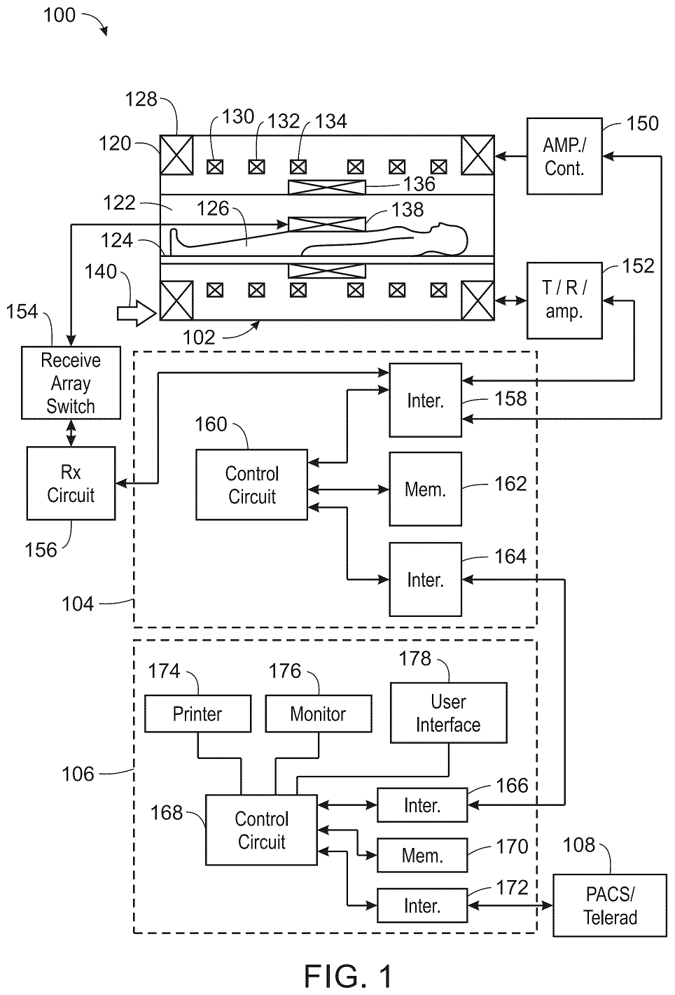

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, magnetic resonance data may be acquired, followed by on- or off-site processing and evaluation. While the magnetic resonance imaging system 100 may include any suitable scanner or detector, in the illustrated embodiment, the system 100 includes a full body scanner 102 having a housing 120 through which a bore 122 is formed.

A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B₀. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to generate a variety of data for training a deep learning-based segmentation model as described below. In certain embodiments, the memory circuit 170 may store one or more neural networks (e.g., deep learning-based segmentation network for generating brain images (or skull stripped images), lesion core masks, bleeds and calcification masks, healthy brain masks (i.e., lacking brain abnormality)). In certain embodiments, the disclosed techniques may occur on a separate computing device having processing circuitry and memory circuitry.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

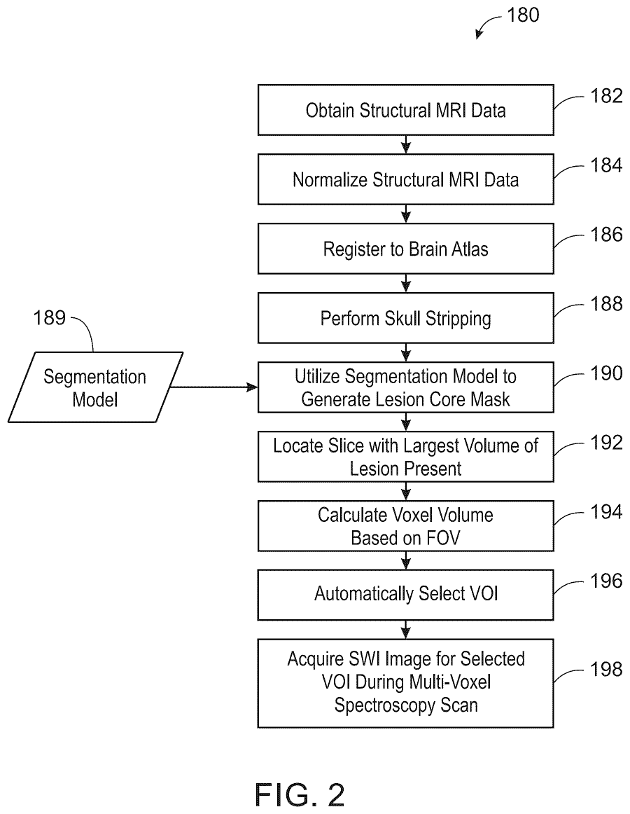

FIG. 2 illustrates a flow diagram of a method 180 for performing multi-voxel spectroscopy. One or more steps of the method 180 may be performed by processing circuitry of the magnetic resonance imaging system 100 in FIG. 1 or a remote computing device. One or more of the steps of the method 180 may be performed simultaneously or in a different order from the order depicted in FIG. 2. One or more (and in some cases) all of the steps of the method 180 may be performed automatically.

The method 180 includes obtaining structural magnetic resonance imaging data (e.g., T1-weighted image, T2-weighted image, FLAIR image, T1 weighted image post contrast) of a brain of a subject acquired with a magnetic resonance imaging scanner (block 182). The method 180 also includes normalizing the structural magnetic resonance imaging data (block 184). In certain embodiments, normalization of the structural magnetic resonance imaging data includes performing N4 bias field correction to correct low frequency intensity non-uniformity (e.g., bias or gain field) present in the structural magnetic resonance imaging data. The method 180 further includes registering the structural magnetic resonance imaging data to a standard brain atlas (e.g., to have a common coordinate system) (block 186). The method 180 even further includes performing skull stripping on the structural magnetic resonance imaging data (i.e., to remove or extract skull and other-non-brain tissues) to generate a brain image (or skull stripped image) (block 188).

The method 180 further includes utilizing a trained deep learning-based segmentation model 189 to generate a lesion core mask from the brain image (block 190). In certain embodiments, in the lesion core mask, the gadolinium enhanced tumor or lesion is labeled and the necrotic and nonenhancing tumor core and edema are also separately labeled. The method 180 even further include locating a slice with largest volume of lesion present in the lesion core mask (block 192). The method 180 still further includes calculating a voxel volume (e.g., for prescription) that avoids aliasing from the slice based on a field of view (FOV) (block 194). The method 180 also includes automatically selecting a volume of interest (VOI) in the brain having both the lesion and normal brain tissue for a multi-voxel spectroscopy scan by the magnetic resonance imaging scanner based on the skull stripped brain image, the lesion core mask, and the voxel volume (block 196). For example, as described in greater detail below, a volume of interest selection algorithm may be utilized to select the volume of interest. The method 180 include acquiring susceptibility-weighted (SWI) image data of the brain of the subject for the volume of interest (e.g., prescribed volume of interest) during a multi-voxel spectroscopy scan with the magnetic resonance imaging scanner (block 198).

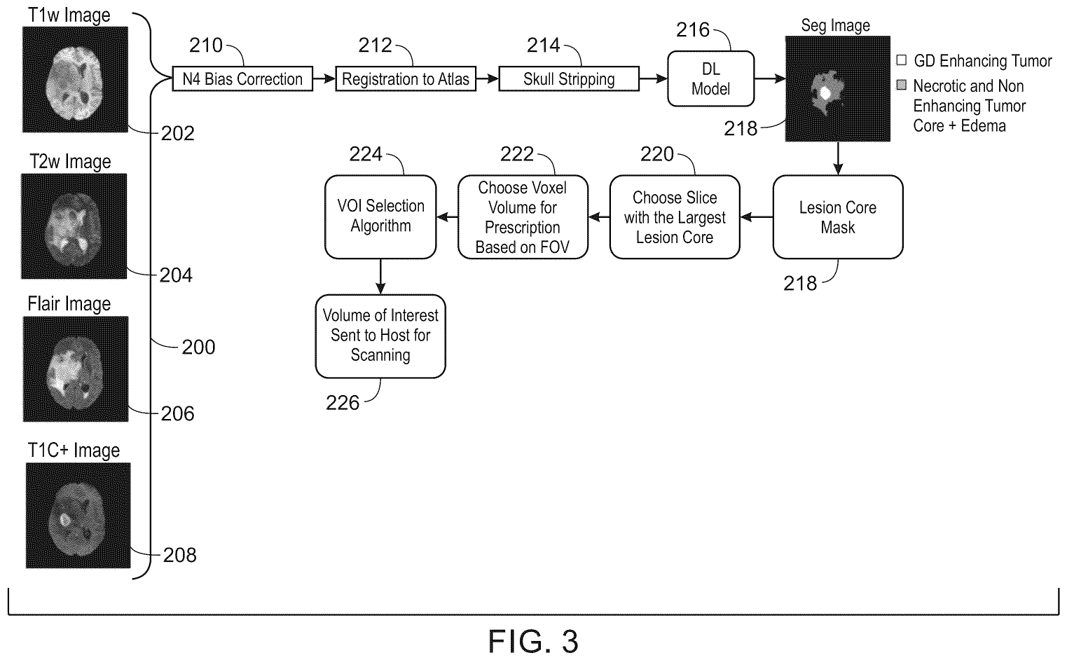

FIG. 3 is a schematic diagram illustrating the process for prescribing a volume of interest for multi-voxel spectroscopy. As indicated in FIG. 3, structural magnetic resonance imaging data 200 of a brain of a subject acquired with a magnetic resonance imaging scanner is obtained. As depicted, the structural magnetic resonance imaging data 200 includes T1-weighted image volume 202, a T2-weighted image volume 204, a FLAIR image volume 206, and a T1-weighted image volume post contrast 208. As indicated by reference numeral 210, N4 bias field correction is performed on the structural magnetic resonance imaging data 200 to correct low frequency intensity non-uniformity (e.g., bias or gain field) present in the structural magnetic resonance imaging data 200. As indicated by reference numeral 212, the normalized structural magnetic resonance imaging data is registered to a standard brain atlas (e.g., to have a common coordinate system). As indicated by reference numeral 214, skull stripping is performed on normalized and registered structural magnetic resonance imaging data 200 to generate a brain image (or skull stripped mask).

The brain image is inputted into a deep learning-based segmentation model 216 which outputs a lesion core mask 218. As depicted in the lesion core mask 218, the gadolinium enhanced tumor or lesion is labeled and the necrotic and nonenhancing tumor core and edema are also separately labeled. As indicated by reference numeral 220, a slice is located or chosen with largest volume of lesion present in the lesion core mask. As indicated by reference numeral 222, a voxel volume (e.g., for prescription) that avoids aliasing from the slice is chosen or calculated based on a field of view (FOV) (block 194). A volume of interest (VOI) selection algorithm is then utilized to automatically select a volume of interest in the brain having both the lesion and normal brain tissue for a multi-voxel spectroscopy scan by the magnetic resonance imaging scanner based on the skull stripped brain image, the lesion core mask, and the voxel volume as indicated by reference numeral 226. The selected volume of interest (or prescribed volume of interest) is then sent to a host for a multi-voxel spectroscopy scan (e.g., by the magnetic resonance scanner).

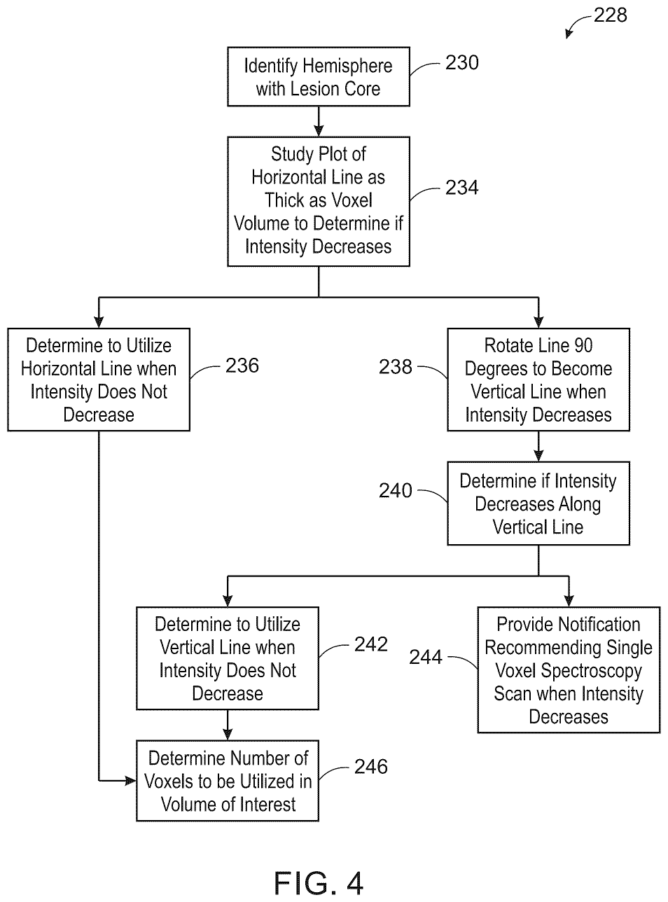

FIG. 4 illustrates a flow diagram of a method 228 for selecting a volume of interest (e.g., by a volume of interest selection algorithm) in the brain having both the lesion and normal brain tissue for a multi-voxel spectroscopy scan. One or more steps of the method 228 may be performed by processing circuitry of the magnetic resonance imaging system 100 in FIG. 1 or a remote computing device. One or more of the steps of the method 228 may be performed simultaneously or in a different order from the order depicted in FIG. 4. One or more (and in some cases) all of the steps of the method 228 may be performed automatically.

The method 228 includes identifying in which hemisphere (e.g., left or right) of the brain that a core of the lesion is located utilizing the brain mask (e.g., brain mask determined in block 188 of the method 188 in FIG. 2) (block 230). The method 228 also includes determining in the brain tissue mask whether to utilize a horizontal volume of interest or vertical volume of interest as indicated by reference numeral 232. In particular, in determining in the brain tissue mask whether to utilize the horizontal volume of interest or the vertical volume of interest, the method 228 includes studying a plot of a horizontal line as thick as the voxel volume (e.g., voxel volume determined in block 194 of the method 188 in FIG. 2) that both originates from the brain tissue mask from the hemisphere of the brain where the core of the lesion is located and passes through a center of the core until an end of the brain tissue mask to determine if intensity decreases along the horizontal line (block 234). In certain embodiments, in determining in the brain tissue mask to utilize the horizontal volume of interest or the vertical volume of interest, the method 228 includes determining that the horizontal volume of interest can be utilized when the intensity does not decrease along the horizontal line (block 236). In certain embodiments, in determining in the brain tissue mask to utilize the horizontal volume of interest or the vertical volume of interest further, the method 236 includes rotating the horizontal line 90 degrees to become a vertical line when the intensity does decrease along the horizontal line (block 238). In certain embodiments, in determining in the brain tissue mask to utilize the horizontal volume of interest or the vertical volume of interest, the method 228 includes determining if the intensity decreases along the vertical line (block 240). In certain embodiments, in determining in the brain tissue mask to utilize the horizontal volume of interest or the vertical volume of interest, the method 228 includes determining that the vertical volume of interest can be utilized when the intensity does not decrease along the vertical line. (block 242). In certain embodiments, the method 228 includes providing a notification (e.g., on the operator console or any device utilized by a user) recommending a single voxel spectroscopy scan of the brain instead of a multi-voxel spectroscopy scan when the intensity decreases along both the horizontal line and the vertical line (block 244).

The method 228 also includes determining a number of voxels to be utilized in the volume of interest (block 246). In particular, the number of voxels is determined in both x- and y-directions. The x- and y-directions are relative to the line (whether the line is in a vertical or horizontal orientation). For example, the x-direction is along the line while the y-direction is cross-wise to the line. To identify the number of voxels along the y-direction, the length of the drawn or plotted line and the voxel volume are both utilized. To identify the number of voxels along the x-direction, initially 1 voxel is chosen and continuously increased by another voxel until no dips in intensity are seen for that thickness of the line. The thickness of the line is number of voxels times the voxel volume.

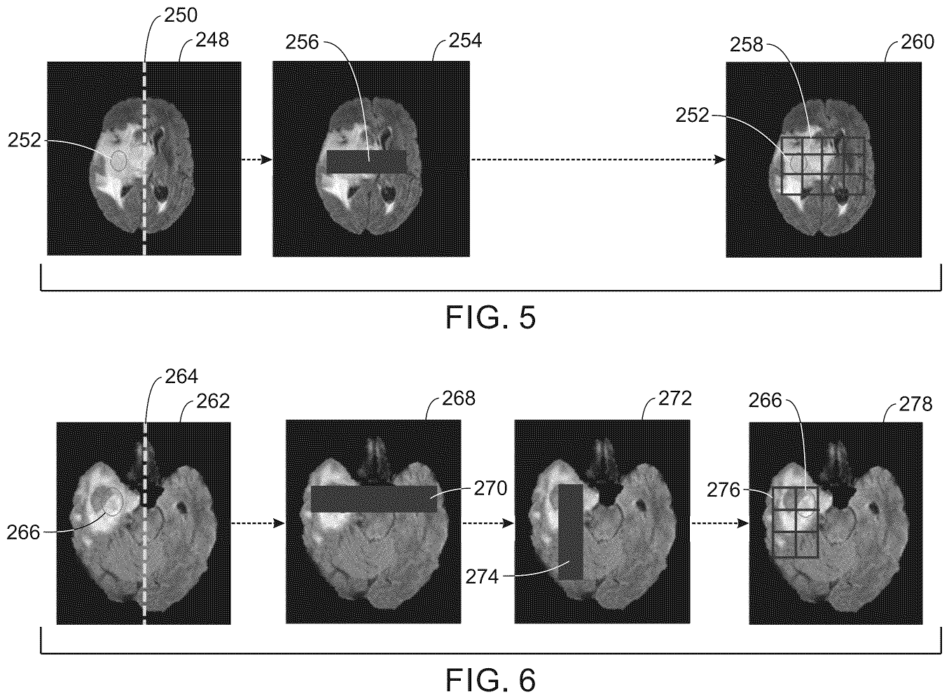

FIGS. 5 and 6 illustrate the utilization of the volume of interest selection algorithm. FIG. 5 illustrates utilization of a volume of interest selection algorithm (e.g., utilizing a horizontal volume). In brain image 248, dashed line 250 demarcates the two hemispheres of the brain and ellipsis 252 indicates the lesion core. In the brain image 254, a horizontal line 256 as thick as the voxel volume that both originates from the brain image from the hemisphere of the brain where the core of the lesion is located and passes through a center of the core until an end of the brain image is plotted to determine if intensity decreases along the horizontal line. In this case, the intensity did not decrease and a horizontal volume of interest is utilized and a number of voxels determined to be utilized in the volume of interest 258 as marked on the brain image 260.

FIG. 6 illustrates utilization of a volume of interest selection algorithm (e.g., utilizing a vertical volume). In brain image 262, dashed line 264 demarcates the two hemispheres of the brain and ellipsis 266 indicates the lesion core. In the brain image 268, a horizontal line 270 as thick as the voxel volume that both originates from the brain image from the hemisphere of the brain where the core of the lesion is located and passes through a center of the core until an end of the brain image is plotted to determine if intensity decreases along the horizontal line. In this case, the intensity did decrease along the horizontal line 270 due to air. Thus, as indicated in brain image 272, the horizontal line is rotated 90 degrees to become a vertical line 274 and the line is studied to see if the intensity decreases along the vertical line 274. In this case, a vertical volume of interest is utilized and a number of voxels determined to be utilized in the volume of interest 276 as marked on the brain image 278.

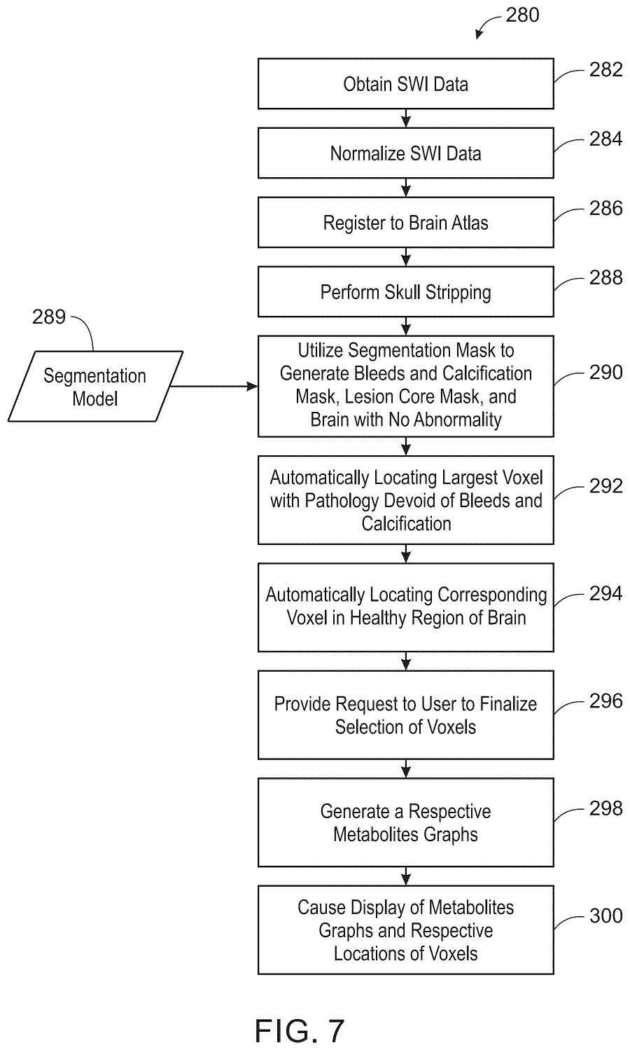

FIG. 7 illustrates a flow diagram of a method 280 for post-processing for multi-voxel spectroscopy. One or more steps of the method 280 may be performed by processing circuitry of the magnetic resonance imaging system 100 in FIG. 1 or a remote computing device. One or more of the steps of the method 280 may be performed simultaneously or in a different order from the order depicted in FIG. 7. One or more (and in some cases) all of the steps of the method 280 may be performed automatically.

The method 280 includes obtaining susceptibility-weighted image data of a brain of a subject acquired during a multi-voxel spectroscopy scan with a magnetic resonance imaging scanner (block 282). The method 280 also includes normalizing the susceptibility-weighted image data (block 284). In certain embodiments, normalization of the susceptibility-weighted image data includes performing N4 bias field correction to correct low frequency intensity non-uniformity (e.g., bias or gain field) present in the susceptibility-weighted image data. The method 280 further includes registering the susceptibility-weighted image data to a standard brain atlas (e.g., to have a common coordinate system) (block 286). The method 280 even further includes performing skull stripping on the susceptibility-weighted image data (i.e., to remove or extract skull and other-non-brain tissues) to generate a brain image (or skull stripped image) (block 288).

The method 280 further includes utilizing a trained deep learning-based segmentation model 289 to generate a bleeds and calcification mask. The lesion core mask, and brain mask with no abnormality from the brain image from the segmentation model 189 are re-used in this method. The method 280 even further includes automatically locating within a prescribed volume of interest the largest voxel with pathology devoid of bleeds and calcification based on both the lesion core mask and the bleeds and calcification mask, wherein the prescribed volume of interest has both a lesion and normal brain tissue (block 292). The method 280 still further includes automatically locating within the prescribed volume of interest a corresponding voxel (e.g., contralateral voxel) of a healthy region of the brain (also devoid of bleeds and calcification) based on both the brain mask with no abnormality and the bleeds and calcification mask (block 294).

The method 280 yet further includes providing a request to user to finalize selection of voxels including the largest voxel with pathology devoid of bleeds and calcification and the corresponding voxel of the healthy region (block 296). In certain embodiments, the request includes an option presented to the user to edit or to add more voxels. The method 280 further includes generating, upon finalization of selection of voxels, respective metabolites graphs for both the largest voxel with pathology devoid of bleeds and calcification and the corresponding voxel of the healthy region (block 298). In certain embodiments, the method 280 includes causing display of both the respective metabolites graphs and respective locations of both the largest voxel with pathology devoid of bleeds and calcification and the corresponding voxel of the healthy region on a reference image of the brain of the subject utilized for prescription of the volume of interest for the multi-voxel spectroscopy scan (block 300).

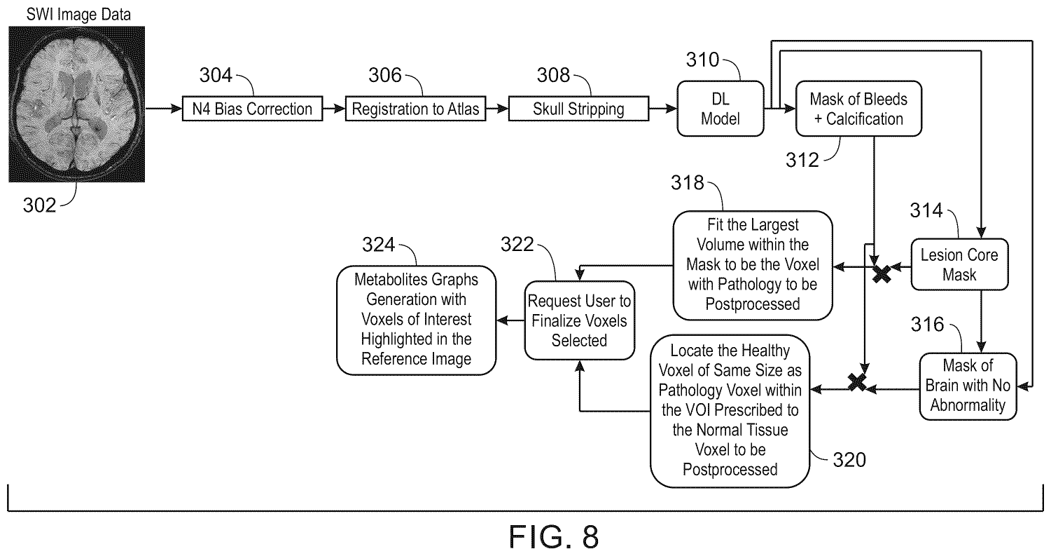

FIG. 8 is a schematic diagram illustrating the process for post-processing for multi-voxel spectroscopy. As indicated in FIG. 8, susceptibility-weighted image data 302 of a brain of a subject acquired during a multi-spectroscopy scan (e.g., for a prescribed volume interest as determined in the method 180 in FIG. 2) with a magnetic resonance imaging scanner is obtained. As indicated by reference numeral 304, N4 bias field correction is performed on the susceptibility-weighted image data 302 to correct low frequency intensity non-uniformity (e.g., bias or gain field) present in the susceptibility-weighted image data 302. As indicated by reference numeral 306, the normalized susceptibility-weighted image data is registered to a standard brain atlas (e.g., to have a common coordinate system). As indicated by reference numeral 308, skull stripping is performed on normalized and registered susceptibility-weighted image data to generate a brain image (or skull stripped image).

The brain image is inputted into a deep learning-based segmentation model 310 which outputs a bleeds and calcification mask 312, a lesion core mask 314, and a mask of the brain with no abnormality 316 (i.e., healthy brain mask). The lesion core mask 314 is utilized in generating the mask of the brain with no abnormality 316. As indicated by reference numeral 318, the largest voxel with pathology devoid of bleeds and calcification is automatically located within a prescribed volume of interest based on both the lesion core mask and the bleeds and calcification mask, wherein the prescribed volume of interest has both a lesion and normal brain tissue. In particular, the lesion core mask 314 is multiplied with the bleeds and calcification mask 312. As indicated by reference numeral 318, a corresponding voxel (e.g., contralateral voxel) of a healthy region of the brain (also devoid of bleeds and calcification) is automatically located within the prescribed volume of interest based on both the brain mask with no abnormality and the bleeds and calcification mask. In particular, the mask of the brain with no abnormality 316 is multiplied with the bleeds and calcification mask 312

As indicated by reference numeral 322, a request is provided to the user to finalize selection of voxels including the largest voxel with pathology devoid of bleeds and calcification and the corresponding voxel of the healthy region. In certain embodiments, the request includes an option presented to the user to edit or to add more voxels. As indicated by reference numeral 324, upon finalization of selection of voxels, respective metabolites graphs for both the largest voxel with pathology devoid of bleeds and calcification and the corresponding voxel of the healthy region are generated and then displayed along respective locations of both the largest voxel with pathology devoid of bleeds and calcification and the corresponding voxel of the healthy region on a reference image of the brain of the subject utilized for prescription of the volume of interest for the multi-voxel spectroscopy scan.

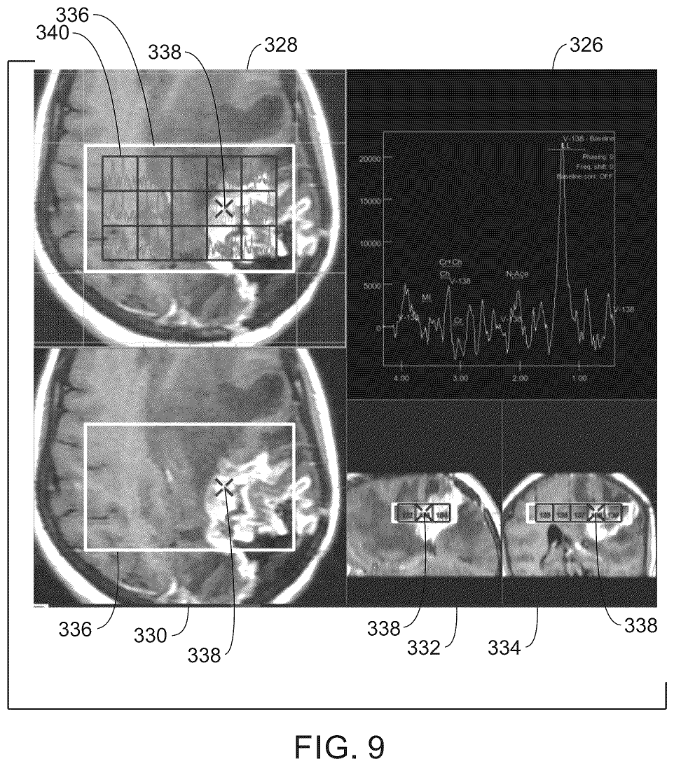

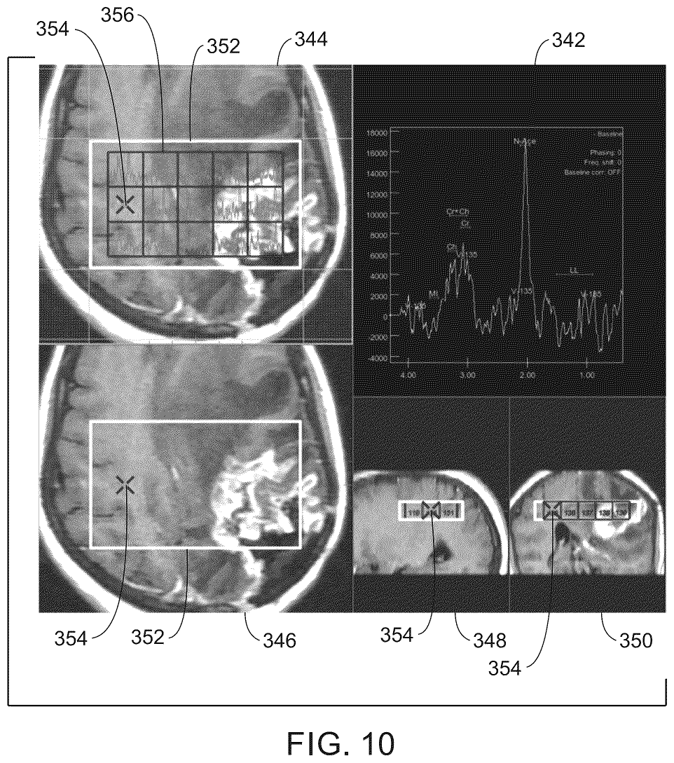

FIGS. 9 and 10 illustrate the post processing automation for multi-voxel spectroscopy. FIG. 9 illustrates display of a metabolites graph 326 and corresponding location for voxel placed in contrast enhanced portion of pathology. Magnetic resonance images 328, 330, 332, and 334 are reference images of the brain of the subject utilized for prescription of the volume of interest for the multi-voxel spectroscopy scan. Magnetic resonance images 328 and 330 are axial views. Magnetic resonance images 330 and 332 are sagittal and coronal views, respectively. The prescribed volume of interest 336 is indicated in Magnetic resonance images 328 and 330. Cross-hair 338 indicates the location of the voxel placed in the contrast enhanced portion of the pathology. Grid 340 denotes the voxels within the volume of interest 336. The prescribe volume of interest 336 includes both the pathology and adjacent or contralateral healthy brain tissue.

FIG. 10 illustrates display of a metabolites graph 342 and corresponding location for a contralateral voxel (relative to the voxel in the pathology FIG. 9) in a healthy brain region. Magnetic resonance images 344, 346, 348, and 350 are reference images of the brain of the subject utilized for prescription of the volume of interest for the multi-voxel spectroscopy scan. Magnetic resonance images 344 and 346 are axial views. Magnetic resonance images 348 and 350 are sagittal and coronal views, respectively. The prescribed volume of interest 352 is indicated in Magnetic resonance images 344 and 346. Cross-hair 338 indicates the location of the voxel placed in the contrast enhanced portion of the pathology. Grid 356 denotes the voxels within the volume of interest 352. The prescribe volume of interest 352 includes both the pathology and adjacent or contralateral healthy brain tissue.

Technical effects of the disclosed subject matter include help the user detect pathology and select a slice for spectroscopy planning. The disclosed embodiments also reduce planning time and avoid unnecessary re-scanning of spectroscopy due to lack of expertise for first time magnetic resonance imaging users. The disclosed embodiments help avoid intra-voxel magnetic field inhomogeneity artifacts by excluding the bone and air-tissue interface while prescribing the spectroscopy volume of interest. The disclosed embodiments enable the most appropriate voxels to be chosen automatically for studying, thus, helping with the magnetic resonance imaging reporting.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing multi-voxel spectroscopy, comprising:
obtaining, at a processor, structural magnetic resonance imaging data of a brain of a subject acquired with a magnetic resonance imaging scanner;
performing, via the processor, skull stripping on the structural magnetic resonance imaging data to generate a skull stripped brain image;
utilizing, via the processor, a trained deep learning-based segmentation model to generate a lesion core mask from the skull stripped brain image;
locating, via the processor, a slice with largest volume of lesion present in the lesion core mask;
calculating, via the processor, a voxel volume that avoids aliasing from the slice based on a field of view;
automatically selecting, via the processor, a volume of interest in the brain having both the lesion and normal brain tissue for a multi-voxel spectroscopy scan by the magnetic resonance imaging scanner based on the skull stripped brain image, the lesion core mask, and the voxel volume.

2. The computer-implemented method of claim 1, wherein automatically selecting the volume of interest comprises identifying, via the processor, in which hemisphere of the brain that a core of the lesion is located utilizing the brain mask.

3. The computer-implemented method of claim 2, wherein automatically selecting the volume of interest further comprises determining, via the processor, in the brain tissue mask whether to utilize a horizontal volume of interest or vertical volume of interest.

4. The computer-implemented method of claim 3, wherein determining in the brain tissue mask whether to utilize the horizontal volume of interest or the vertical volume of interest comprises studying, via the processor, a plot of a horizontal line as thick as the voxel volume that both originates from the brain tissue mask from the hemisphere of the brain where the core of the lesion is located and passes through a center of the core until an end of the brain tissue mask to determine if intensity decreases along the horizontal line.

5. The computer-implemented method of claim 4, wherein determining in the brain tissue mask to utilize the horizontal volume of interest or the vertical volume of interest further comprises determining, via the processor, that the horizontal volume of interest can be utilized when the intensity does not decrease along the horizontal line.

6. The computer-implemented method of claim 4, wherein determining in the brain tissue mask to utilize the horizontal volume of interest or the vertical volume of interest further comprises rotating, via the processor, the horizontal line 90 degrees to become a vertical line when the intensity does decrease along the horizontal line.

7. The computer-implemented method of claim 6, wherein determining in the brain tissue mask to utilize the horizontal volume of interest or the vertical volume of interest further comprises determining, via the processor, if the intensity decreases along the vertical line.

8. The computer-implemented method of claim 7, wherein determining in the brain tissue mask to utilize the horizontal volume of interest or the vertical volume of interest further comprises determining, via the processor, that the vertical volume of interest can be utilized when the intensity does not decrease along the vertical line.

9. The computer-implemented method of claim 7, further comprising providing, via the processor, a notification recommending a single voxel spectroscopy scan of the brain instead of a multi-voxel spectroscopy scan when the intensity decreases along both the horizontal line and the vertical line.

10. The computer-implemented method of claim 3, wherein automatically selecting the volume of interest further comprises determining, via the processor, a number of voxels to be utilized in the volume of interest.

11. The computer-implemented method of claim 1, further comprising causing, via the processor, acquisition of susceptibility-weighted image data of the brain of the subject for the volume of interest during a multi-voxel spectroscopy scan with the magnetic resonance imaging scanner.

12. The computer-implemented method of claim 11, further comprising:
obtaining, via the processor, the susceptibility-weighted image data;
performing, via the processor, skull stripping on the susceptibility-weighted image data to generate a second skull stripped brain image;
utilizing, via the processor, the trained deep learning-based segmentation model to generate a bleeds and calcification mask, a second lesion core mask, and a brain mask with no abnormality from the second skull stripped brain image;
automatically locating, via the processor, within the volume of interest the largest voxel with pathology devoid of bleeds and calcification based on both the second lesion core mask and the bleeds and calcification mask;
automatically locating, via the processor, within the volume of interest a corresponding voxel of a healthy region of the brain based on both the brain mask with no abnormality and the bleeds and calcification mask;
providing, via the processor, a request to user to finalize selection of voxels including the largest voxel with pathology devoid of bleeds and calcification and the corresponding voxel of the healthy region; and
generating, via the processor, upon finalization of selection of voxels, respective metabolites graphs for both the largest voxel with pathology devoid of bleeds and calcification and the corresponding voxel of the healthy region.

13. The computer-implemented method of claim 12, further comprising causing, via the processor, display of both the respective metabolites graphs and respective locations of both the largest voxel with pathology devoid of bleeds and calcification and the corresponding voxel of the healthy region on a reference image of the brain of the subject utilized for prescription of the volume of interest for the multi-voxel spectroscopy scan.

14. A computer-implemented method for post-processing for multi-voxel spectroscopy, comprising:
obtaining, at a processor, susceptibility-weighted image data of a brain of a subject acquired during a multi-voxel spectroscopy scan with a magnetic resonance imaging scanner;
performing, via the processor, skull stripping on the susceptibility-weighted image data to generate a skull stripped brain image;
utilizing, via the processor, a trained deep learning-based segmentation model to generate a bleeds and calcification mask, a lesion core mask, and a brain mask with no abnormality from the skull stripped brain image;
automatically locating, via the processor, within a prescribed volume of interest the largest voxel with pathology devoid of bleeds and calcification based on both the lesion core mask and the bleeds and calcification mask, wherein the prescribed volume of interest has both a lesion and normal brain tissue;
automatically locating, via the processor, within the prescribed volume of interest a corresponding voxel of a healthy region of the brain based on both the brain mask with no abnormality and the bleeds and calcification mask;
providing, via the processor, a request to user to finalize selection of voxels including the largest voxel with pathology devoid of bleeds and calcification and the corresponding voxel of the healthy region; and
generating, via the processor, upon finalization of selection of voxels, respective metabolites graphs for both the largest voxel with pathology devoid of bleeds and calcification and the corresponding voxel of the healthy region.

15. The computer-implemented method of claim 14, further comprising causing, via the processor, display of both the respective metabolites graphs and respective locations of both the largest voxel with pathology devoid of bleeds and calcification and the corresponding voxel of the healthy region on a reference image of the brain of the subject utilized for prescription of the prescribed volume of interest for the multi-voxel spectroscopy scan.

16. The computer-implemented method of claim 14, wherein the request comprises an option presented to the user to edit or to add more voxels.

17. A system for performing multi-voxel spectroscopy, comprising:
a memory encoding processor-executable routines; and
a processor configured to access the memory and to execute the processor-executable routines, wherein the processor-executable routines, when executed by the processor, cause the processor to:
obtain structural magnetic resonance imaging data of a brain of a subject acquired with a magnetic resonance imaging scanner;
perform skull stripping on the structural magnetic resonance imaging data to generate a first skull stripped brain image;
utilize a trained deep learning-based segmentation model to generate a first lesion core mask from the first skull stripped brain image;
locate a slice with largest volume of lesion present in the lesion core mask;
calculate a voxel volume that avoids aliasing from the slice based on a field of view;
automatically select a volume of interest in the brain having both the lesion and normal brain tissue for a multi-voxel spectroscopy scan by the magnetic resonance imaging scanner based on the skull stripped brain image, the lesion core mask, and the voxel volume.

18. The system of claim 17, wherein the processor-executable routines, when executed by the processor, further cause the processor to acquire susceptibility-weighted image data of the brain of the subject for the volume of interest during a multi-voxel spectroscopy scan with the magnetic resonance imaging scanner.

19. The system of claim 18, wherein the processor-executable routines, when executed by the processor, further cause the processor to:
obtain the susceptibility-weighted image data;
perform skull stripping on the susceptibility-weighted image data to generate a second skull stripped brain image;
utilize the trained deep learning-based segmentation model to generate a bleeds and calcification mask, a second lesion core mask, and a brain mask with no abnormality from the second skull stripped brain image;
automatically locate within the volume of interest the largest voxel with pathology devoid of bleeds and calcification based on both the second lesion core mask and the bleeds and calcification mask;
automatically locate within the volume of interest a corresponding voxel of a healthy region of the brain based on both the brain mask with no abnormality and the bleeds and calcification mask;
provide a request to user to finalize selection of voxels including the largest voxel with pathology devoid of bleeds and calcification and the corresponding voxel of the healthy region; and
generate, upon finalization of selection of voxels, respective metabolites graphs for both the largest voxel with pathology devoid of bleeds and calcification and the corresponding voxel of the healthy region.

20. The system of claim 19, wherein the processor-executable routines, when executed by the processor, further cause the processor to cause display of both the respective metabolites graphs and respective locations of both the largest voxel with pathology devoid of bleeds and calcification and the corresponding voxel of the healthy region on a reference image of the brain of the subject utilized for prescription of the volume of interest for the multi-voxel spectroscopy scan.
